(19) United States
(12) Patent Application Publication
(10) Pub. No.: US 2022/0378430 A1
(43) Pub. Date: Dec. 1, 2022

(54) A TUBULAR TISSUE TRANSFORMER

(71) Applicant: Avasa Limited, Auckland (NZ)

(72) Inventors: Walimuni Nandoun Akbho Mendis ABEYSEKERA, Auckland (NZ); Han Sang KIM, Auckland (NZ); Jon MATHY, Auckland (NZ)

(21) Appl. No.: 17/755,786

(22) PCT Filed: Nov. 4, 2020
(86) PCT No.: PCT/NZ2020/050141

§ 371 Date: May 6, 2022

(30) Foreign Application Priority Data
Nov. 8, 2019 (NZ) 758894

(51) Int. Cl. A61B 17/11

(52) U.S. Cl. CPC A61B 17/11; A61B 2017/1107; A61B 2017/1121

(57) ABSTRACT

A tubular tissue transformer (TTT) for a tubular tissue structure. The tubular tissue transformer has a plurality of leaves and a plurality of retainers on each of the plurality of leaves. Each retainer retains the tubular tissue structure on the respective leaf. Also provided are a coupling system to coupling tubular tissue structures together and tools and methods for attaching and widening otubular tissue structures.

### FIELD

This invention relates generally to a tubular tissue transformer for tubular tissue structures and related tools and methods.

### BACKGROUND

In surgical procedures, tissue structures may be coupled together to form an anastomosis. Traditionally, this involves manually suturing the tissue structures together, which may be time-consuming, risky, demanding and may require extensive training and high precision.

Devices for assisting the coupling of tissue structures may retain the tissue structure by way of large fixed pins, to which the tissue structure is attached one at a time. However, some tissue structures may tear if subjected to the strain involved in attaching them to the pins. This may be particularly problematic for tissue structures that are relatively thick-walled or inflexible such as arteries.

Tissue structures can be everted prior to coupling to each other to ensure good contact between inner surfaces of the structures for healing. In some situations, this may cause damage to the tissue structure by deforming it excessively and preventing anastomosis. This may be particularly problematic for tissue structures that are relatively thick-walled or inflexible such as arteries. In some situations, it may be difficult to provide surfaces of the right geometry to maintain good contact between the inner surfaces of the coupled tissue structures.

### SUMMARY

According to one exemplary embodiment there is provided a tubular tissue transformer for a tubular tissue structure, the tubular tissue transformer comprising:

a plurality of leaves; and

a plurality of retainers on each of the plurality of leaves, each retainer configured to retain the tubular tissue structure on the respective leaf.

According to another exemplary embodiment there is provided a method of attaching a tubular tissue structure to a tubular tissue transformer, the method comprising:

pressing a portion of the tubular tissue structure to simultaneously retain it at a plurality of locations.

According to another exemplary embodiment there is provided a tubular tissue transformer for a tubular tissue structure, the tubular tissue transformer comprising:

a plurality of leaves configured to retain an everted portion of the tubular tissue structure; and

a bushing configured to be movable between a first position and a second position and configured to support an outer surface of the everted portion of the tubular tissue structure when in the second position.

According to another exemplary embodiment there is provided a method comprising:

everting a portion of a tubular tissue structure; and supporting the outer surface of an everted portion of the tubular tissue structure over a surface that curves outwardly away from the centre of the everted portion.

According to another exemplary embodiment there is provided a tool for widening a portion of a tubular tissue structure, the tubular tissue structure retained on a tubular tissue transformer about an opening of the tubular tissue transformer, the tool comprising:

a tapered portion for insertion into the opening to widen the opening, thereby widening the portion of the tubular tissue structure.

According to another exemplary embodiment there is provided a tool for widening a portion of a tubular tissue structure, the tubular tissue structure retained on a tubular tissue transformer about an opening of the tubular tissue transformer, the tool comprising:

an expandable portion for insertion into the opening and expanding in the opening to widen the opening, thereby widening the portion of the tubular tissue structure.

According to another exemplary embodiment there is provided a method of widening a portion of a tubular tissue structure, the method comprising:

retaining a portion of the tubular tissue structure with a first diameter;

while the portion of the tubular tissue structure is retained, deforming the portion of the tubular tissue structure to a second diameter, greater than the first diameter; and

retaining the portion of the tubular tissue structure with the second diameter.

According to another exemplary embodiment there is provided a tubular tissue transformer for a tubular tissue structure, the tubular tissue transformer comprising:

one or more leaves located about a passage of the tubular tissue transformer and configured to retain a portion of the tubular tissue structure; and

a bushing configured to be locatable at least partly within the passage;

wherein the bushing is at least partly plastically deformable to radially expand the one or more leaves and retain the leaves in their radially expanded state.

According to another exemplary embodiment there is provided a system for coupling tubular tissue structures, the system comprising:

a first tubular tissue transformer having one or more retainers for retaining a portion of a tubular tissue structure, the one or more retainers being located at one or more retainer locations about the retained portion of the tubular tissue structure;

a second tubular tissue transformer having one or more retainers for retaining a portion of a tubular tissue structure, the one or more retainers being located at one or more retainer locations about the retained portion of the tubular tissue structure;

a first coupling device; and

a second coupling device configured to couple to the first coupling device;

wherein the first and second coupling devices are configured to couple the retained portions of the tubular structures and maintain predetermined rotational offsets between the one or more retainer locations of the first tubular tissue transformer and the one or more retainer locations of the second tubular tissue transformer, the rotational offsets being offsets about a longitudinal axis through the coupling devices, when the retained portions are coupled.

According to another exemplary embodiment there is provided an attachment tool comprising:

a deformable surface configured to press a portion of a tubular tissue structure against a plurality of retainers of a tubular tissue transformer to attach the portion of the tubular tissue structure to the tubular tissue transformer.

Embodiments may be implemented according to any of the dependant claims.

It is acknowledged that the terms “comprise”, “comprises” and “comprising” may, under varying jurisdictions, be attributed with either an exclusive or an inclusive meaning. For the purpose of this specification, and unless otherwise noted, these terms are intended to have an inclusive meaning — i.e., they will be taken to mean an inclusion of the listed components which the use directly references, and possibly also of other non-specified components or elements.

Reference to any document in this specification does not constitute an admission that it is prior art, validly combinable with other documents or that it forms part of the common general knowledge.

### BRIEF DESCRIPTION OF THE DRAWINGS

The accompanying drawings which are incorporated in and constitute part of the specification, illustrate embodiments of the invention and, together with the general description of the invention given above, and the detailed description of embodiments given below, serve to explain the principles of the invention, in which:

FIG. 1A is a perspective view of a tubular tissue transformer according to one example in a non-expanded rest state.

FIG. 1B is a perspective view of an exemplary alternative to a tubular tissue transformer.

FIG. 2A is a perspective view of the tubular tissue transformer of FIG. 1 in an expanded state.

FIG. 2B is a perspective view of the tubular tissue transformer of FIG. 1B in an expanded state.

FIG. 3A is an exploded view of the tubular tissue transformer of FIGS. 1A and 2A.

FIG. 3B is an exploded view of the tubular tissue transformer of FIGS. 1B and 2B.

FIG. 4 is a cross section of a tubular tissue transformer according to one example.

FIG. 5 is a perspective view of a tubular tissue transformer according to one example and a tubular tissue structure located in a passage of the tubular tissue transformer.

FIG. 6 is a perspective view of a tubular tissue transformer according to one example and a tubular tissue structure retained on the tubular tissue transformer.

FIG. 7 is a perspective view of a tubular tissue transformer according to one example and a tubular tissue structure everted on the tubular tissue transformer.

FIG. 8A is a perspective view of a system for coupling tubular tissue structures according to one example.

FIG. 8B is a perspective view of an exemplary alternative system for coupling tubular tissue structures.

FIG. 9A is an exploded view of the system of FIG. 8A.

FIG. 9B is an exploded view of an exemplary alternative to the system of FIG. 8. FIG. 9C is an exploded view of the system of FIG. 8B.

FIG. 10 is a perspective view of a tubular tissue transformer, a coupling device according to one example and a tubular tissue structure.

FIG. 11 is a perspective view of the system for coupling tubular tissue structures of FIG. 8 and tubular tissue structures.

FIG. 12 is a cross-sectional view of a tubular tissue structure inserted into a tubular tissue transformer according to one example.

FIG. 13 is a cross-sectional view a tool operating on the inserted tubular tissue structure of FIG. 12 according to one example.

FIG. 14 is a cross-sectional view of the tool of FIGS. 12 and 13 operating on a tubular tissue structure to attach it to the tubular tissue transformer according to one example.

FIG. 15 is a cross-sectional view of tubular tissue transformer in a non-expanded, rest state and a tubular tissue structure retained on the tubular tissue transformer according to one example.

FIG. 16 is a cross-sectional view of the tubular tissue transformer of FIG. 15 in an expanded state and the tubular tissue structure of FIG. 15 everted on the tubular tissue transformer according to one example.

FIG. 17 is a cross-sectional view of a tool operating on a tubular tissue transformer and a tubular tissue structure retained on the tubular tissue transformer according to one example.

FIG. 18 is a cross-sectional view of the tool of FIG. 17 operating on a bushing of the tubular tissue transformer of FIG. 17 to deform the bushing according to one example.

FIG. 19 is a partially cross-sectional view of a system for coupling tubular tissue structures and tubular tissue structures according to one example and tubular tissue structures.

FIG. 20 is a partially cross-sectional view of the system and tubular tissue structures of FIG. 19 with tubular tissue transformers of the system engaged with coupling devices of the system according to one example.

FIG. 21 is a cross-sectional view of the system and tubular tissue structures of FIGS. 19 and 20 with the coupling devices and tubular tissue structures coupled together according to one example.

### DETAILED DESCRIPTION

The present application relates to a tubular tissue transformer (TTT) that has leaves, a bushing and retainers. Each leaf has more than one retainer. The retainers may allow a tubular tissue structure to be simultaneously attached to more than one retainer, rather than requiring them to be attached one at a time. This may simplify and speed up attachment of the tissue structure.

The bushing is also designed to cause eversion of the tissue structure by expanding the leaves and support the outer surface of the everted tissue structure. This may provide a large, well-supported surface of the tissue structure for coupling to another tissue structure and may reduce damage to the tissue structure during eversion.

The present application also relates to tools and methods for assisting attachment of the tissue structure and widening the tissue structure and a system for coupling tubular tissue transformers and tubular tissue structures together.

The following terminology will be used throughout:
Tubular tissue transformer (TTT) a device that transforms a tissue structure to facilitate anastomosis. It may optionally also retain the tissue structure; evert the tissue structure, and/or change the diameter of the tissue structure. It may optionally also retain the integrity of the tissue structure during one or more of these processes. References to a tubular tissue transformer or TTT throughout the specification and claims should be understood to refer to such a device.
Tubular tissue structure a part of body of a human or other animal formed from tissue and being generally tubular in shape with an inner lumen. Examples include vessels such as veins, arteries, lymphatic vessels, ureters, pancreatic ducts, bowel and other ducts.
Leaf a part of an object that extends another part of the object and has a significant width transverse to its length in at least one location along the length.
Bushing a member located about the inner perimeter of a passage or opening.
Evert in the context of a tubular tissue structure means turn outwards such that an inner surface of the tissue structure about the lumen is accessible for contact. Derived terms such as eversion and everted have meanings consistent with this.
Anastomosis a circumferential connection between tubular tissue structures.

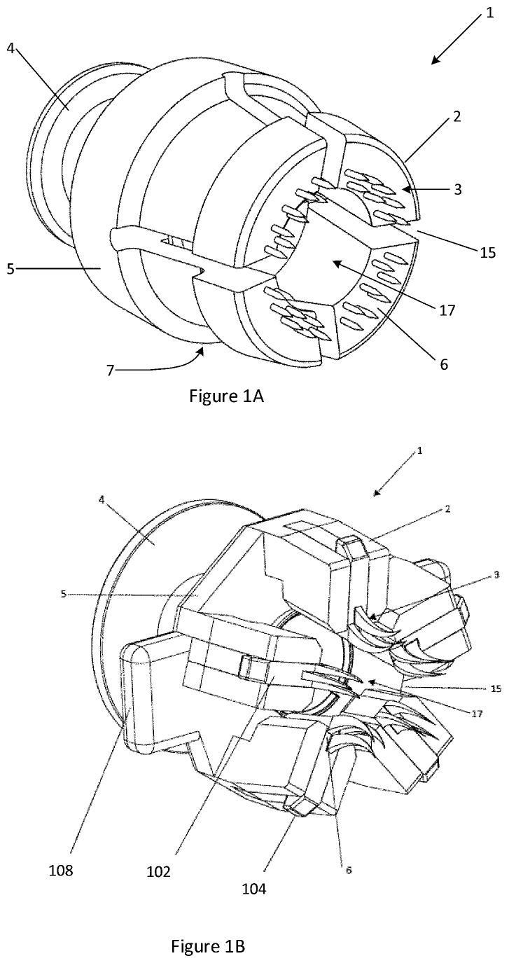

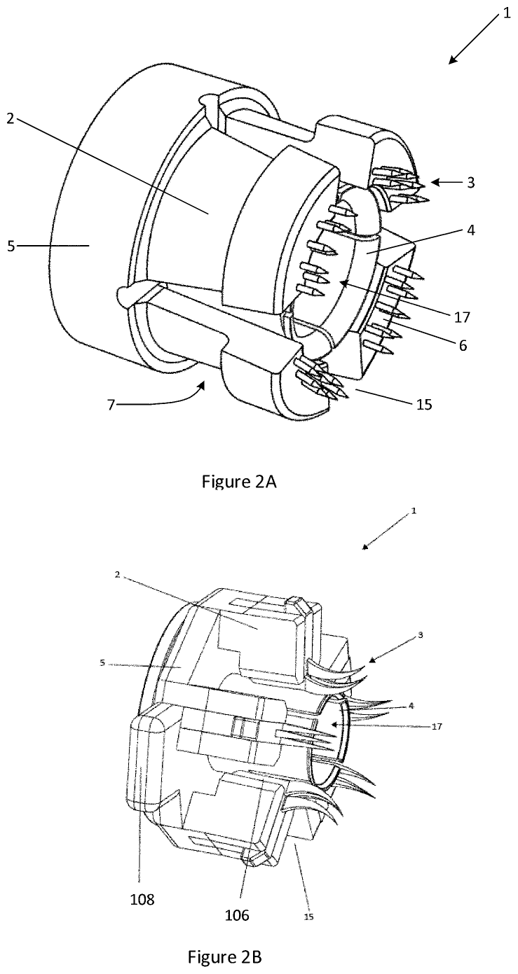

An exemplary tubular tissue transformer (TTT) 1 is shown in FIGS. 1A, 2A and 3A. This device 1 has leaves 2, with retainers 3 on each of the leaves 2. In this example, the TTT 1 also includes a bushing 4. In FIG. 1A, the device 1 is in a first configuration with the leaves 2 in their rest positions and the bushing 4 in a non-advanced position. FIG. 2A depicts the same device 1 in a second configuration with the leaves 2 in an expanded configuration and the bushing 4 in an advanced position.

Each of the leaves 2 in this example has a number of retainers that retain an attached tubular tissue structure. Having more than one retainer on each leaf 2 may traditionally be considered disadvantageous on a device that required attachment of the tissue structure to each retainer individually. However, the retainers on each leaf of the present TTT 1 are designed to simultaneously attach to and retain the tissue structure in one step without the need for individual attachment to each retainer. This may reduce the time, skill and expertise required to attach the tissue structure to a retainer.

The retainers may be suction ports, pins, grippers or other elements that can attach to the tissue structure. In the example of FIG. 1A, the retainers are pins 3. The pins 3 may be straight or may curve outwardly. By outwardly, it is meant at an angle away from a longitudinal axis 19 passing through the TTT 1, which is generally aligned with the direction of extension of the leaves 2. The retainers could also be a combination of some straight pins and some curved pins. In some uses, straight pins may be easier to push into the tissue structure. In some uses, curved pins may retain the tissue structure better and reduce the chance of it detaching from the TTT 1. Straight pins may extend at an angle away from the longitudinal axis 19. There may be a combination of straight pins at different angles.

The lengths of the pins 3 in this example are between 0.2 mm and 1.5 mm, for example between 0.5 mm and 1.2 mm. Different length pins 3 may be suitable for different applications, such as for different tissue structures. For example, short pins may be better suited for attachment to small or thin-walled structures whereas longer pins may be better suited to large or thick-walled structures. The TTT 1 could have pins of various lengths on each leaf 2. Having pins of various lengths on each leaf may reduce the amount of preparation of the outer layer (or adventitia) of the tubular tissue structure prior to attachment. This may also reduce the time required for a coupling procedure.

The large number of retainers may allow the tissue structure to be attached at a large number of points around the tissue structure, which may reduce the stress on each point of attachment. A large number of attachment points may also reduce the required strength of connection to each individual retainer, which may avoid the need for relatively destructive retainers like large pins that make large holes in the tissue structure and may potentially cause significant damage to the tissue structure. It may also allow relatively small, closely grouped retainers to be used which may be able to simultaneously attach to a tissue structure that is pressed onto them. Different numbers of retainers may be suitable depending on the nature and size of the tissue structure, size and type of retainer and number of leaves 2. For example, thick-walled or relatively inflexible tissue structures may require more retainers per leaf 2, as might large tissue structures. Similarly, larger numbers of retainers may be needed when individual retainers are smaller. Larger numbers of retainers on each leaf 2 may be needed if the TTT 1 has a small number of leaves 2. In one example, there are between 2 and 10 retainers on each leaf 2. In one example, there are at least 8 retainers in total on the TTT 1. In the example of FIG. 1A, there are 8 retainers, in the form of pins 3, on each leaf 2. In this example, there are a total of 32 retainers on the TTT 1.

The retainers may be arranged in one or more rows on each leaf 2. This may allow more retainers to fit on the retention surface 6 of each leaf 2. In the example of FIG. 1A, there are two rows of pins 3 on each leaf 2; an outer row of 5 pins and an inner row of 3 pins. In order to fit more retainers on each leaf, the retainers may be placed close together, for example with between 0.2 mm and 0.5 mm between neighbouring retainers.

Different numbers of leaves may be suitable for different applications. A greater number of leaves may allow for more even distribution of forces on the tissue structure, especially during any expansion or eversion that the tissue structure may undergo. A smaller number of leaves may be easier for an operator to manipulate. The number of leaves may be at least four or at least five. In the example of FIG. 1A, the TTT 1 has four leaves 2. In an alternative example, there may be a single leaf in place of the plurality of leaves 2. In this example, the leaf may be generally cylindrical or frusto-conical with a changeable perimeter. The perimeter of such a leaf may change by deforming, stretching or coiling and uncoiling.

The leaves 2 may be located about a passage 17 through the TTT 1. In use, a tissue structure may be located in the passage 17 and retained on the leaves 2 about the opening of the passage 17. The passage 17 would be sized to be able to accommodate the tissue structure.

The leaves 2 are flexible to widen and narrow. A portion of the leaves 2 can move outwardly away from, or inwardly towards, the longitudinal axis 19. In the example of FIGS. 1A, 2A and 3A, the leaves 2 extend from a ring or base 5. The leaves 2 and ring 5 together from a single integral body 10. The distal ends of the leaves 2 can move towards and away from the axis 19 to expand or contract the opening of the passage 17. This may allow the diameter of the opening to be changed to assist attachment of the tissue structure to the TTT 1 or to another tissue structure. The outward flexing of the leaves 2 may also be useful for everting the tissue structure, as will be described in more detail with reference to FIGS. 6 and 7.

To flex the leaves 2 inwards, an operator may grip the leaves 2, for example with forceps, and squeeze them. This allows the diameter of the opening to be reduced to make attachment of the tissue structure easier. To assist with this process, the leaves 2 may be provided with features that make them easier to grip. In the example of FIG. 1A, the TTT 1 has a groove 7 formed in the leaves 2 that can receive the ends of forceps and help prevent them slipping off the TTT 1.

In alternative examples with a different construction from that shown in FIG. 1A, a different portion of the leaves 2 may be expanded or narrowed. For example, if the tissue structure is retained at a portion that is between the ends of the leaves 2, the leaves 2 may be expanded or narrowed at this portion.

The leaves 2 may be elastically flexible throughout the typical range of flexure experienced in use such that they return to their original configuration after they are released.

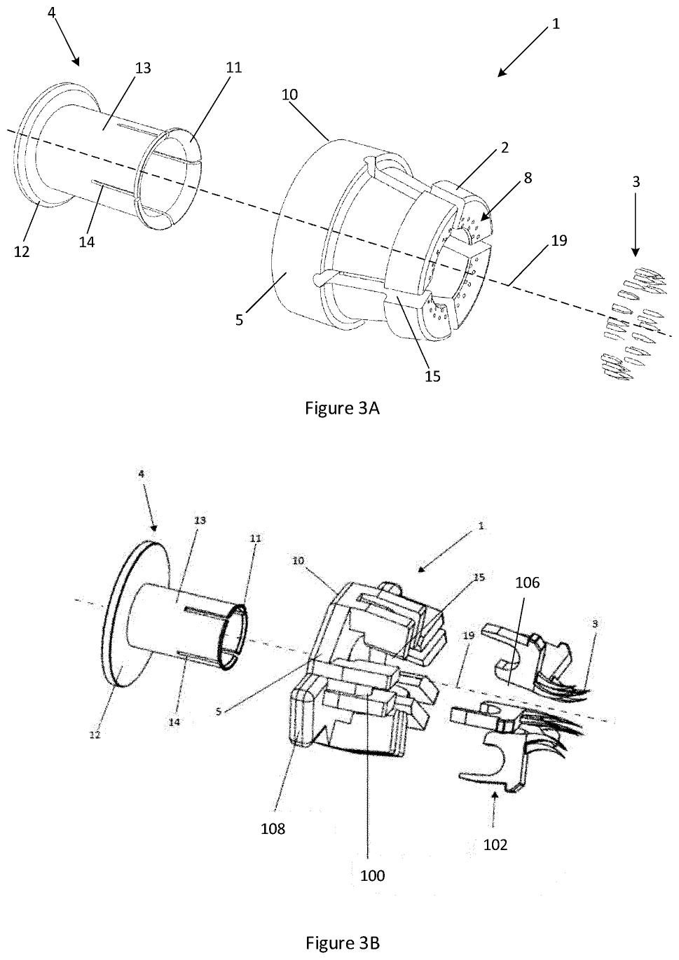

In the exploded view of FIG. 3A, the body 10, retainers (in the form of pins) 3 and bushing 4 are shown separately. Holes 8 in the leaves 2 are provided to receive the pins 3 of this example. The pins 3 are arranged about a circle, as are the leaves 2. The bushing 4 can be seen in more detail in this view.

The bushing 4 includes a substantially cylindrical body 13. At the front of the bushing 4 (i.e. the end nearest the distal ends of the leaves 2), the bushing 4 is formed into a support face 11. This support face 11 is provided to support the outer surface of the tissue structure in use. The support face 11 may be formed from a widened portion of the bushing 4. The widened portion may also bear against the inner surfaces of the leaves 2 to drive them outwardly as the bushing 4 is advanced from the first, rearward position to the second, advanced position. The widened portion may also engage with the leaves 2 to resist the bushing 4 being moved out of the advanced position back towards the rearward position. For example, it may extend beyond the ends of the leaves 2 such that a rear surface of the widened portion is in contact with the ends of the leaves 2, thereby resisting being pulled back past the leaves 2. Alternatively, there may be a groove or asymmetric ramp on the inner surface of the leaves 2 which the widened portion engages with to resist being pulled back out of the groove or past the steep side of the ramp.

The support face 11 may be formed from a flange extending outward from, and at a sharp angle to, the body 13 of the bushing 4, for example at 90°. Alternatively, the support face 11 may be formed from a “flared” portion that curves outwardly from the body 13. A tubular tissue structure can be supported over this surface with an outward curve due to the support face curving outwardly away from the centre of the supported portion of the tubular tissue structure. The support face 11 may curve outwardly by between 10° and 120°, or between 30° and 90°. The support face 11 may curve outwardly with a radius of curvature selected based on the properties of the tissue structure to be supported. Some tissue structures may suffer unacceptable damage if turned outwards too tightly. In such cases, it may be advantageous to select the radius of curvature to be greater than a value which would be likely to cause unacceptable damage to the tissue structure. Arteries, for example, have relatively thick and inelastic walls compared with other tissue structures such as veins, and may be unacceptably damaged if turned outwards too tightly. In some examples, the radius of curvature is greater than 0.2 mm.

The bushing 4 also may include a flange 12 or other feature to prevent it being advanced beyond the second, advanced position. The flange 12 can bear against the rear surface of the ring 5, or another part of the body 10, to prevent the bushing 4 moving forward beyond the advanced position. Alternatively, the bushing 4 may include a widened portion to form a friction fit with an opening of the body 10 of the TTT 1; a bayonet-type fitting to fit into a complementary fitting of the body 10 of the TTT 1; or adhesive to adhere to the body 10 of the TTT 1. If the bushing 4 includes a widened portion, bayonet-type fitting or adhesive, this may additionally act to resist the bushing 4 being moved out of the second position towards the first position, in addition to or instead of the widened portion forming the support face 11.

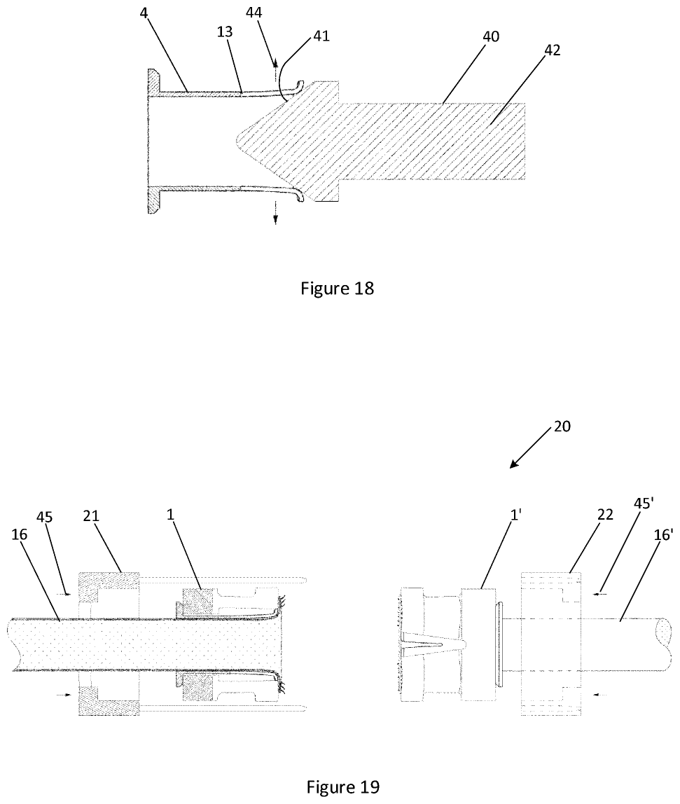

The bushing 4 may also have gaps 14 in the body 13 to allow widening of the bushing 4, as will be detailed with reference to FIGS. 17 and 18. Notwithstanding the gaps 14, the bushing 4 may be substantially circular in cross section. By substantially circular, it is meant that the bushing 4 forms more than 50%, more than 75%, more than 85%, or preferably more than 90% of a full circle, while noting that the circle referred to may not be perfectly circular in a practical implementation.

The bushing 4 may also cause flexure of the leaves 2. In this example, the bushing 4 is provided within the passage 17 and configured to move along the passage 17. As shown in FIGS. 1A and 2A, the bushing 4 can be moved between a first, rearward position (FIG. 1A) and a second, advanced position (FIG. 2A). With the bushing 4 in the rearward position, the leaves 2 are not expanded—i.e. they are in a “rest” configuration—as shown in FIG. 1A. When the bushing 4 moves to the advanced position, the outer edge of the widened portion that forms the support face 11 bears on the inner surfaces of the leaves 2 and forces them into the radially expanded configuration of FIG. 2A and retains them in that configuration. Alternatively, the bushing 4 may have another portion, separate from the widened portion that forms the support face 11, to bear on the leaves 2 to expand them and/or retain them in the expanded configuration.

The bushing 4 may be at least partly plastically deformable. This allows it to be deformed upon application of a force and retain the deformed shape after the force is removed. The bushing 4, or part thereof, may be formed from a material with appropriate deformation properties depending on the application. For example, the material can be selected such that it can undergo plastic deformation at typical forces applied by an operator in a widening procedure (detailed with respect to FIGS. 17 and 18) but retain its shape (i.e. be rigid) at typical forces applied by the leaves 2 and retained portion of the tissue structure before or after the widening procedure. One suitable material would be metal, such as stainless or surgical steel, titanium alloy, or cobalt-chromium. Another suitable material would be a polymer, such as a polytetrafluoroethylene/silicone composite.

Parts of the TTT 1 may be transparent in order to allow an operator to see the tissue structure during use. In particular, the bushing 4 and/or one or more of the leaves 2 may be transparent.

In one example, the TTT 1 may be provided with suction ports. The suction ports alone or in combination with pins 3 can constitute the retainers. In one example, the suction ports are provided at the ends of the pins 3.

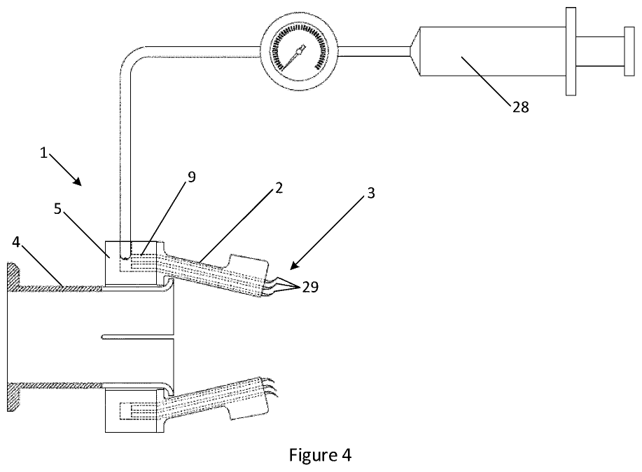

FIG. 4 shows an example in which the retainers are pins 3 having suction ports 29 at their ends. The suction ports 29 are connected to a source of low pressure via suction lines 9. In the example of FIG. 4, the suction lines 9 pass through respective pins 3 and leaves 2 and couple to the source of low pressure in the region of the ring 5. The source of low pressure in this example is a syringe 28 that creates a partial vacuum in the suction lines 9 when its plunger is withdrawn. Alternatively, the source of low pressure could be a vacuum pump or similar.

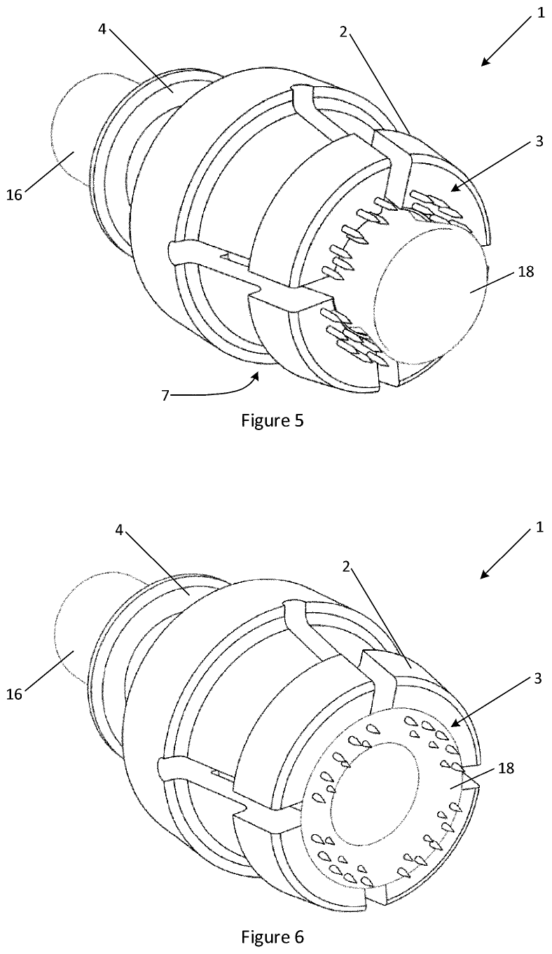

FIGS. 5 to 7 depict the TTT 1 in use with a tubular tissue structure 16 in various states.

In FIG. 5, the tissue structure 16 is located in the passage. The tissue structure in this example has been cut and a portion 18 near the cut end extends out of the passage 17 into the region of the retainers, which are pins 3 in this example. In this state, the bushing 4 is not advanced and the leaves 2 are in their rest positions.

In FIG. 6, the tissue structure 16 has been attached to the retainers 3 at portion 18. As can be seen in FIG. 6, the portion 18 is attached to the retainers at many points arranged generally in a circle and retained on the device 1. In this state, the bushing 4 is not advanced and the leaves 2 are in their rest positions. In this position, the portion 18 of the tissue structure 16 is not completely everted. Depending on the range of motion of the leaves 2 and the angle at which they attach to the tissue structure 16 in the retracted configuration, the portion 18 of the tissue structure 16 may be partially everted or not everted at all.

In FIG. 7, the bushing 4 has been advanced to the advanced position. The leaves 2 have been expanded outwardly. The portion 18 of the tissue structure 16 has thereby been everted more than in the configuration of FIGS. 5 and 6. The portion 18 of the tissue structure 16 may be turned outwards through up to 90°, approximately 90°, or greater than 90° and need not be fully turned “inside out”. In one example, the portion 18 of the tissue structure 16 is turned outwards through approximately 90°. An eversion of 90° may be optimal in some situations to present a large area of the inner surface of the tubular tissue structure 16 for coupling to another structure without turning the tissue structure 16 outwards more than is necessary.

Although not visible in FIG. 7, the support face of the bushing 4 is located near the ends of the leaves 2. In this position, the support face of the bushing 4 is in contact with the outer surface of the everted portion 18 of the tissue structure 16 to support it such that it forms a wide, substantially circular surface suitable for apposition to another tissue structure to form an anastomosis. The ends of the leaves 2 may also form support faces for the everted portion of the tissue structure in this state. In this example, the support faces 6 of the leaves 2 are located near, and at a small angle to, the support face 11 of the bushing 4 so that the leaves 2 and bushing 4 cooperate to provide a composite support face. The support faces 6 of the leaves 2 may be at an angle of less than 45°, less than 30°, or less than 15° to the support face of the bushing 4 in this configuration. In an alternative example, the leaves 2 may provide the entire support face without contribution from the bushing 4.

It can be seen from FIG. 3A that the support face 11 of the bushing 4 covers substantially an entire circle, with only small gaps 14. Even when expanded, these gaps are less than the separation 15 between adjacent leaves 2, providing more support than the support faces 6 of the leaves 2 alone would. In this way, the support face 11 of the bushing helps ensure a large, evenly-supported surface about substantially the whole circumference of the everted portion 18. By substantially the whole circumference, it is meant more than 50%, more than 75%, more than 85%, or preferably more than 90% of the whole circumference. This may help form a good seal between two tissue structures when coupled to form an anastomosis. Due to the outward curve of the support face 11 of the bushing 4 away from the centre of the everted portion, the everted portion 18 of the tissue structure 16 is supported such that it also curves outwardly. It is supported in this shape by the support face 11 in contact with its outer surface. As already noted, this may help avoid damage to the tissue structure 16.

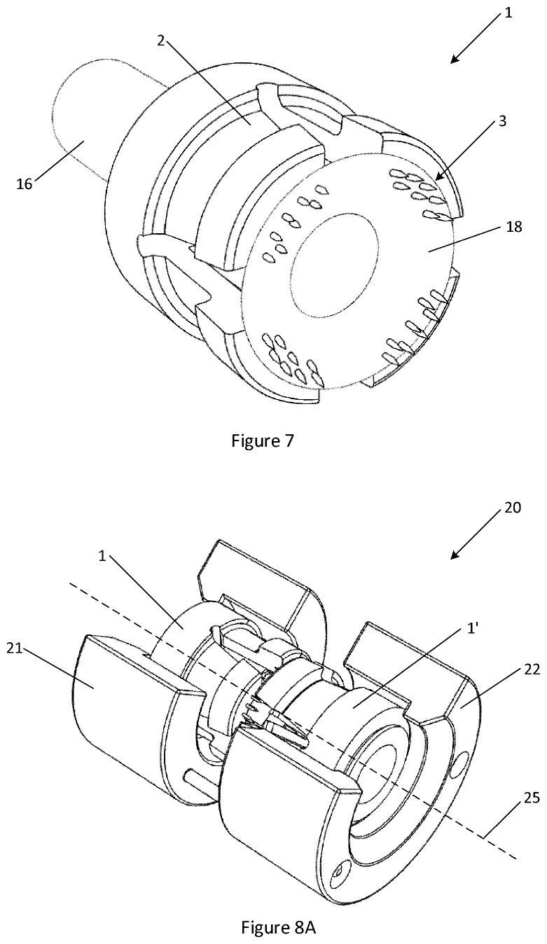

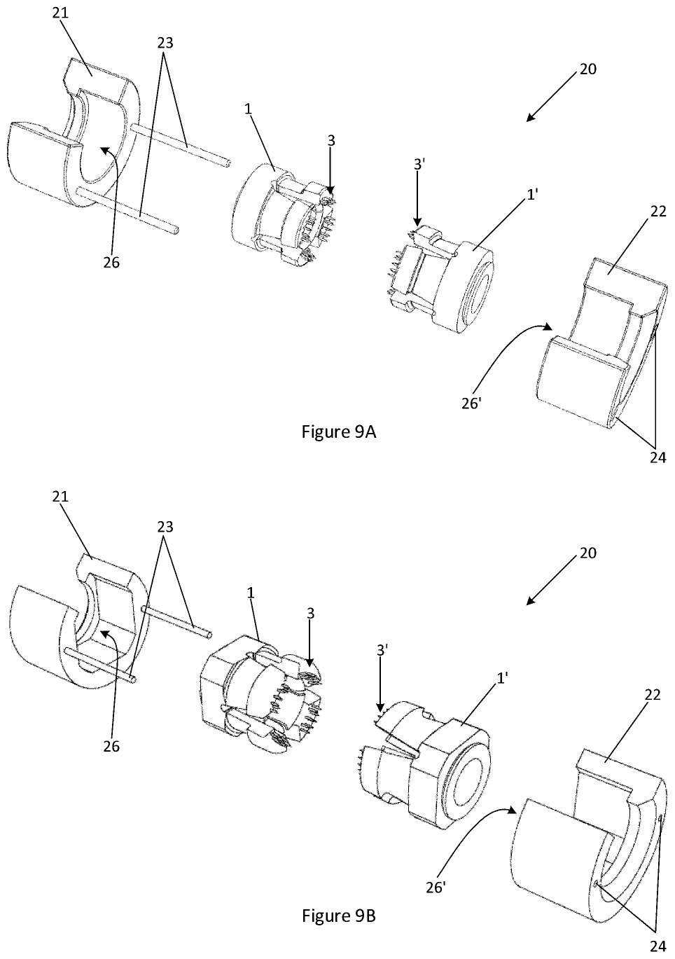

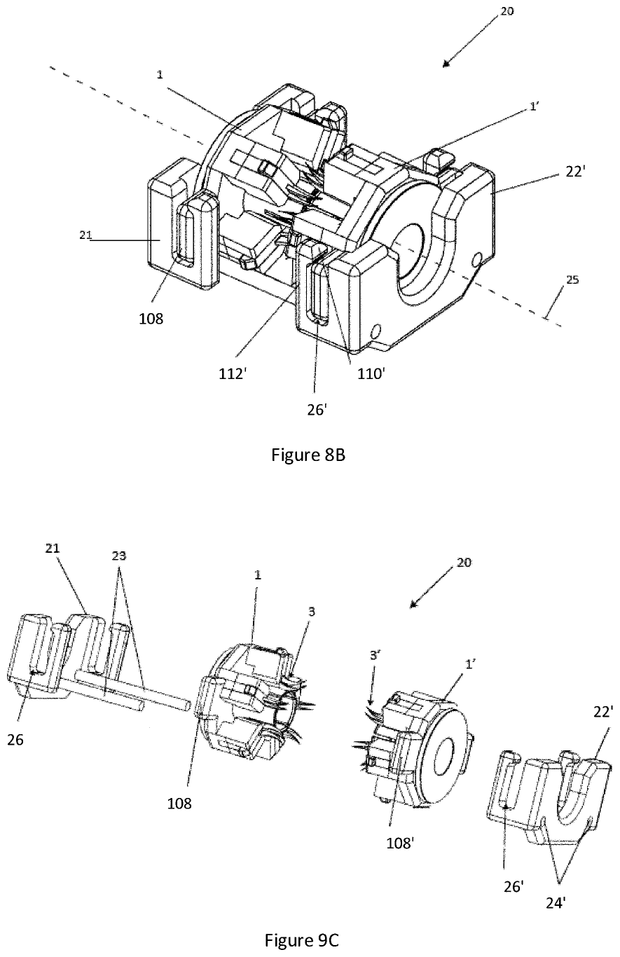

FIG. 8A shows a system 20 for coupling tubular tissue structures including a first TTT 1, a second TTT 1′, a first coupling device 21 and a second coupling device 22.

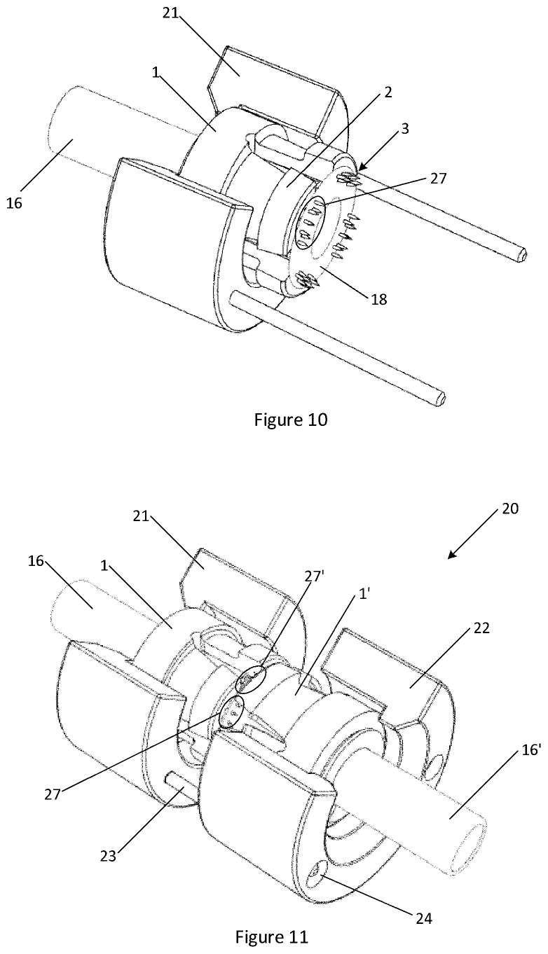

FIG. 9A depicts the system in exploded view, showing the first coupling device, second coupling device, first TTT 1 and second TTT 1′ separately. FIG. 9B depicts an alternative system in exploded view. FIG. 10 shows the first coupling device 21 and first TTT 1 in more detail.

The first and second TTTs 1, 1′ may be the TTTs described with reference to FIGS. 1A, 2A, 3A and 4 to 7 or may be different TTTs. The TTTs 1, 1′ each retain a portion of a tubular tissue structure at at least one respective retention location 27 about the tissue structure. In one example, each of the TTTs 1, 1′ retains the tissue structure at more than one retention locations 27. In the example of FIG. 8, the TTTs 1′, la each have 4 leaves 2, with each leaf 2 having a retention location 27 corresponding to an area covered by a number of pins 3, 3′.

The first and second coupling devices 21, 22 can be brought together to bring the everted portions of the tissue structures together in apposition and coupled together to couple the tissue structures to each other. The coupling devices 21, 22 ensure that the retention locations 27, 27′ of the retention devices are offset from each other when coupled together. This may help ensure a good, even seal around the coupling interface by “filling in” the gaps between one TTT's retention locations with the other device's retention locations. This may also prevent or reduce the likelihood of one device's retainers clashing with the other device's retainers. For example, if the retainers are pins, the predetermined offset prevents the TTTs′ pins from coming into contact with each other. If the pins were to come into contact, this could prevent the retained portions of the tissue structures being brought together well enough to form a good seal.

The coupling devices 21, 22 include alignment features to ensure that they only couple to each other in one of a discrete set of relative orientations about the longitudinal axis 25. In one example, the alignment features are one or more pins and one or more holes for receiving the pin(s). Pins can be provided on both or only one of the coupling devices. Correspondingly, holes can be provided on both or only one of the coupling devices. In the example of FIGS. 8A, 9A and 9B, the first coupling device 21 has two pins 23 and the second coupling device 22 has two holes 24. The pins 23 in this example are not equally spaced around the longitudinal axis 25, i.e. the rotational offset between them is not 360°/n, where n is the number of pins. This restricts the coupling devices 21, 22 to only being able to couple to each other in one relative orientation about the longitudinal axis 25. The pins 23 may have features such as teeth or barbs for engaging with the second coupling device 22 at the perimeters of the holes 24. The pins 23 or holes 24 may be provided with adhesive. The pins 23 or holes 24 may taper to provide a friction fit. In one example, the holes 24 are tapered to engage with the pins 23, which have a constant cross section.

Each coupling device also has one or more alignment features to ensure that it retains the respective TTT 1 in one of a discrete set of relative orientations about the longitudinal axis 25. In other words, the TTT cannot be retained in the coupling device at any angle, only at angles that ensure its retainers are offset from the retainers of the other TTT. This allows the coupling device and the respective TTT 1 to mate in one or more predetermined compatible orientations about the axis 25. Each coupling device can have a recess 26, 26′, in which it receives the respective TTT 1, 1′. In one example, the inner surface of the recess 26, 26′ can be non-circular and an outer surface of a portion of the respective TTT 1, 1′ can also be non-circular. The non-circularity of the devices can prevent them from rotating away from a particular relative orientation when the TTT 1, 1′ is received in the recess 26, 26′. In the example of FIG. 9B, the recesses 26, 26′ and TTTs 1, ′1 are polygonal. Additionally or alternatively, other mating formations may be provided that prevent rotation of the TTT 1, 1′ away from a particular relative orientation with respect to the coupling device 21, 22. For example, these may include a pin in one device and a hole, for receiving the pin, in the other device; a ridge in one device and a groove, for receiving the ridge, in the other. When such a mating formation is provided, the recess 26, 26′ and the outer portion of the TTT 1, 1′ may be circular. In the example of FIGS. 8 and 9A, the recesses 26, 26′ are generally circular in cross section, although each does not complete a full circle in this example. More particularly, each recess is approximately ¾-circular in cross section.

In the example of FIGS. 8A and 9A, the coupling devices 21, 22 are generally circular in cross section but do not complete a full circle. In this example, they are approximately ¾-circular. This means that the coupling devices 21, 22 are each open at one side. This may allow the coupling devices 21, 22 to be moved over the tubular tissue structures from the side to locate the tissue structures within central openings of the coupling devices 21, 22. This may be faster and easier than inserting cut ends of the tissue structures longitudinally through a full-circular opening. The opening may also allow the operator to see the TTTs 1, 1′ and the interface between the tissue structures while coupling them.

FIG. 10 shows the first coupling device 21 with the first TTT 1 located in the recess. The first TTT 1 has a tubular tissue structure 16 retained to it at four retention locations 27 about the retained portion 18.

FIG. 11 shows the system in use to couple a first tubular tissue structure 16 and a second tubular tissue structure 16′ to each other. A retention location 27 of the first TTT 1 is shown offset from a retention location 27′ of the second TTT 1′. A pin 23 of the first coupling device 21 is shown inserted into a hole 24 of the second coupling device 22. In this configuration, the system 20 forms a coupling or anastomosis between the two tubular tissue structures 16, 16′.

An alternative example is shown in FIGS. 1B, 2B, 3B, 8B and 9C. In this case the TTT device 1 has five leaves 2. As seen in FIG. 3B each leaf 2 has a recess 100 to receive a hook insert 102. Each hook insert 102 has a number of retainers/hooks 3 to which a tubular structure can be attached similar to the pins 3 in FIG. 1A. This embodiment may make device fabrication and assembly easier, and improve the process of vessel retention. The hook insert 102 can be made of a hard material like stainless steel with sharp hooks 3 machined using wire EDM. The sharp and hard hooks 3 pierce the wall of the artery easily and a-traumatically. The base 106 of the hook insert 102 provides a smooth surface for the bushing to interface with as it advances through the TTT to flex the leaves radially outwards. The leaves 2 to which the hook inserts 102 insert, must however remain deformable so the leaves 2 can flex radially outwards as the bushing 4 advances forwards to evert the vessel. To achieve all this, the hook insert 102 is most easily fabricated as a separate component that can be inserted in a retrograde fashion into the recess 100. In other words, these recesses 100 can function in a similar way to the holes 8 that receive the pins 3 in FIG. 3A.

The hooks 3 may include 3 hooks per hook insert. There may be 1 inner hook, with 2 outer hooks. Each hook may be tapered and/or outwardly curving. Each hook may be 0.5 to 2.0 mm in length. The thickness of each hook can range from 0.05-1.00 mm, for example it may be 0.15 mm.

The hook insert 102 could be kept in place by employing an interference fit type mechanism. Alternatively, there could be a lip at the back end of the hook insert 102 so it snaps into place in the recess 100. In another alternative, a low viscosity adhesive could be employed to create a bond between the hook insert 102 and the recess 100. A combination of all of the above could also be utilized.

The hook insert 102 has a lip 104 on the outer surface to prevent forceps from slipping off the leaf. Forceps are used to position the TTT 1 appropriately over the vessel that it may be used to retain. When a soft tubular tissue structure such as an artery is being attached to the TTT's hooks 3, the user might pick up the vessel using fine forceps and attach it to the hooks 3. To aid vessel retention and minimize vessel strain during this process, the user might flex the leaves 2 radially inwards to bring the hooks 3 closer to the vessel wall by applying a compressive force with forceps. As the compressive force is applied, the lip 104 prevents the forceps from inadvertently slipping off the leading edge and damaging the soft tissue structure. This lip serves the same function as the groove 7 in FIG. 1A. The lip may be approximately 0.2-1.0 mm in height.

In this particular configuration where there are 5 leaves, if the operator were to grip the leaves from the side of the TTT, one forcep tip would be applied against one leaf at the top and the other forcep tip would be applied against two leaves at the bottom. The leaf at the top would be the leaf that undergoes the most inward flex, and the operator would attach the artery to that set of hooks first. The operator would rotate the TTT and sequentially compress each leaf as he/she goes around attaching the artery to the retainers on each leaf.

The base 106 of the hook insert 102 extends slightly more radially inwards than its respective leaf 2 so that it is this base 106 that interfaces with the bushing 4. This surface provides a smoother interface as the bushing 4 advances to radially expand the leaves. As mentioned, the hook insert may be a rigid material such as stainless steel, titanium or a hard plastic. Rigid in this context means in response to the force provided by pushing the bushing 4 through to the final position.

The leaves 2 could be moulded from a deformable plastic. The plastic and the rigidity of each leaf structure may be designed so that a leaf angle (as compared to the longitudinal axis 19) changes by between 2-15°, or approximately 9° as the bushing 4 is advanced. Deformable in this context means in response to the force provided by pushing the bushing 4 through to the final position.

As shown in FIG. 9C this embodiment of the coupling device 21 features a recess 26 to receive to coupling flanges or wings 108 on either side of the TTT 1, and a lip 110 on a front clip 112 of the coupling device 21 that prevents the TTT 1 from sliding out of the recess 26. The TTTs 1 1′ are attached to each end of the vessel and everted, and clipped into each coupling device 21 21′. Then the two coupling devices 21 21′ are brought in close proximity, are rotationally offset (so that the respective leaves and hooks interlock into the spaces between the opposing leaves) and then are permanently coupled by pins 23. Alternatively each TTT hay include the coupling holes 24 24′, and the coupling pins 23 may engage the respective TTTs 1 1′ directly.

This version of the coupling device 21 may allow improved visibility of the recess 26 into which the TTT 1 must fit. The coupling wings 108 may allow the TTT 1 to be inserted from the top (or conversely that the coupling device 21 can be introduced from below) the thereby reducing the total movement needed to mate the TTT 1 with its corresponding coupling device 21. Mating can be achieved using an interference fit, or a snap lock by means of a deformable front clip 112.

The front clip 112 secures the coupling wings 108 in place and prevents anterograde movement. This means the TTT 1 won't slide out of the coupling device or angle away from the central axis 25.

This approach may also reduce the overall coupler length (i.e. when the coupling devices are linked by the pins 23). This is because the central clearance through which the TTT must first pass through is no longer required before undergoing retrograde translation to fit into the recess 26 as in FIG. 9A.

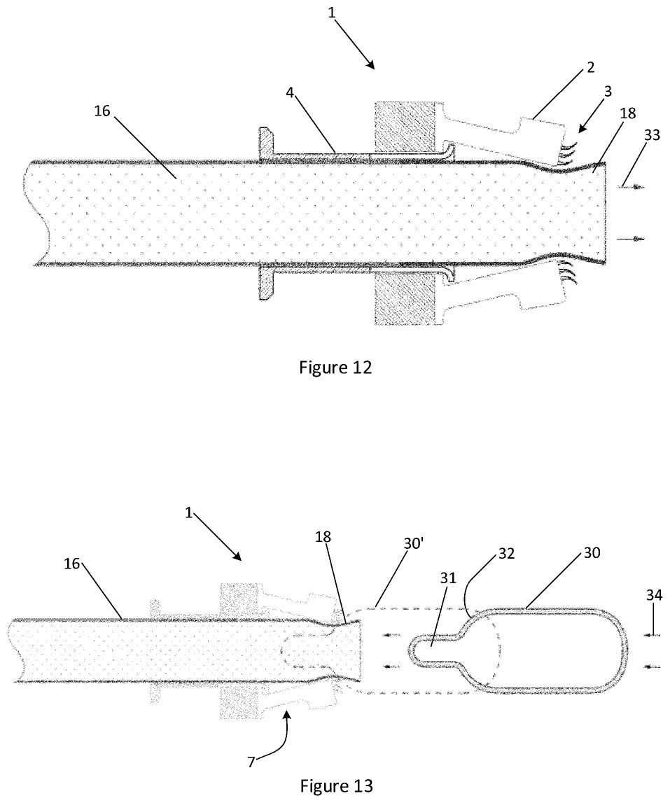

Various methods will now be described with reference to FIGS. 12 to 21. These methods may be performed individually as separate procedures or may be performed together as parts of one procedure.

In FIG. 12, a portion 18 of a tubular tissue structure 16 has been inserted into a passage in a TTT 1 in the direction indicated by the arrows 33. In this example, the inserted portion 18 is the cut end of the tissue structure 16. The TTT 1 in this example is a TTT as described with reference to FIGS. 1 to 8. In such an example, the portion 18 of the tissue structure 16 is passed through the bushing 4 and between the leaves 2.

In FIG. 13, an attachment tool 30 has been brought into contact with the portion 18 of the tissue structure 16 in position 30′. The attachment tool 30 may include a portion, such as tip 31, that is inserted into an opening of the tubular tissue structure 16. The attachment tool 30 has a deformable surface 32 that can press the portion 18 of the tissue structure 16 against a number of retainers at the same time. Before, or while, pressing the portion 18 of the tissue structure 16 against the retainers, the operator may squeeze or otherwise contract the leaves 2 inwardly to bring their ends nearer together. This may make it easier to attach a tissue structure to the TTT 1, especially in the case of narrow or relatively inflexible tissue structures. In one example, the operator grasps the TTT 1 in the groove 7 with forceps and squeezes to contract the leaves 2.

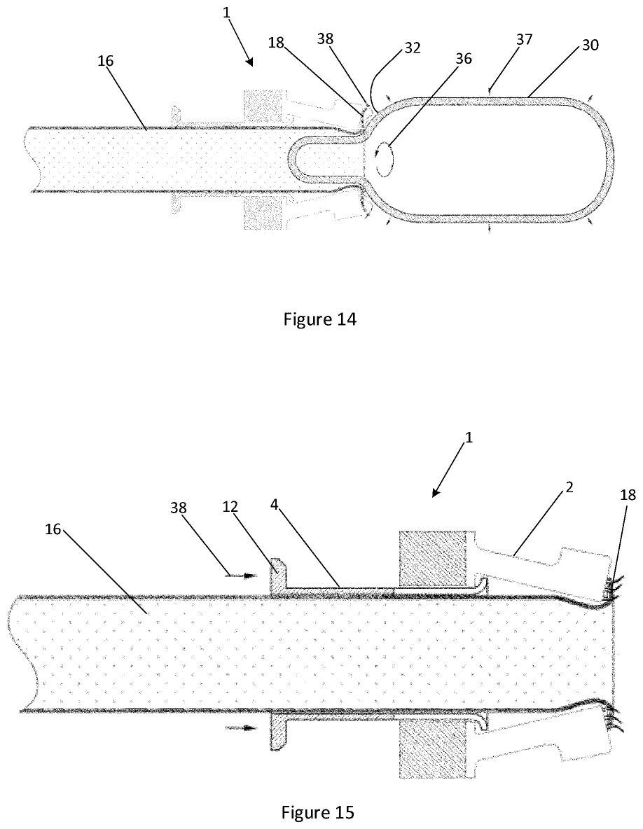

In FIG. 14, the tool 30 is in the position 30′ and the deformable surface 32 has deformed from the state shown in FIG. 13 to make better contact with the portion 18 of the tissue structure 16 and press it against the retainers. The tool 30 may also be rotated, as indicated by the arrow 36, to assist attachment of the portion 18 of the tissue structure 16 around its entire perimeter.

Pressing the portion 18 on the retainers attaches it at a number of locations simultaneously, each location corresponding to one of the retainers. This removes the need for the tissue structure to be attached to the retainers one by one. As indicated by arrows 37, the tissue structure is somewhat turned outwards onto the retainers and attached to them.

The tool 30 may include a fluid, such as air, water or gel, enclosed by the deformable surface 32. In one example, the fluid-filled region can be squeezed or otherwise compressed by an operator in one region in order to cause expansion of the tool at the region in contact with the tissue structure. This can gently press the tissue structure around all or a large part of its perimeter, helping rapid attachment to many retainers at the same time. The deformable surface 32 can be an elastically flexible surface.

In an alternative example, an operator may press the tissue structure without the aid of the tool 30, for example with their finger.

In the example shown in FIGS. 12 to 14, the retained portion 18 of the tissue structure 16 is the portion near the cut end of a tissue structure. In an alternative example, the retained portion may be the area around a slit in the side of a tissue structure. This may enable the TTT 1 to be used as a side coupler for end-to-side coupling of tubular tissue structures.

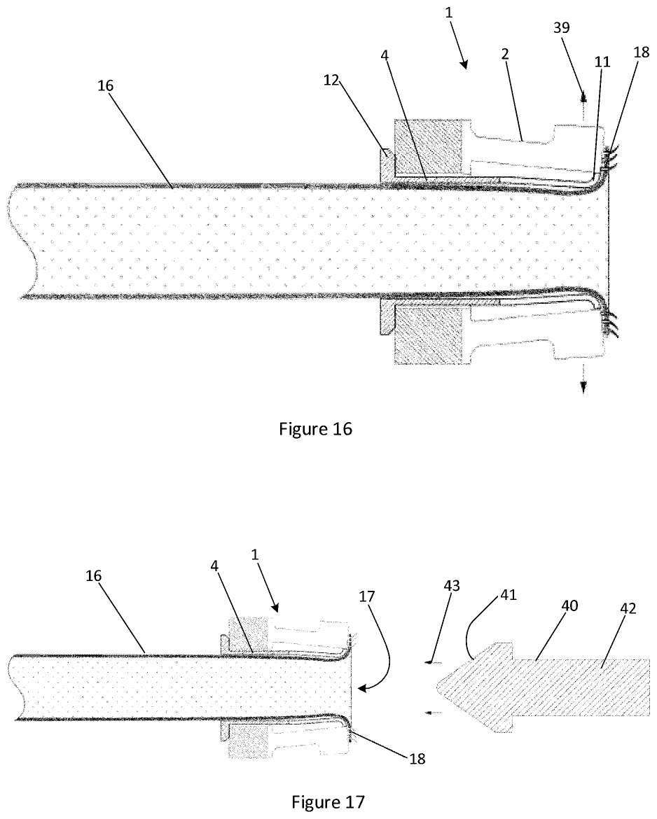

In FIG. 15, the tissue structure 16 is attached to the TTT 1 at portion 18. The bushing 4 is in the first, rearward position and the leaves 2 are in the contracted configuration. By pushing the bushing 4 in the direction indicated by arrows 38, the operator can advance the bushing 4 towards the second, advanced position, shown in FIG. 16.

In FIG. 16, the bushing 4 is in the advanced position and the ends of the leaves 2 have been expanded radially outwards, as indicated by the arrows 39. This expansion has caused eversion of the portion 18 of the tissue structure 16. The everted portion 18 is now supported in the everted state by the support face 11 of the bushing 4. The bushing 4 maintains the everted portion 18 in an outwardly-curved configuration by contacting it at its outer surface with curved support face 11. Thus the TTT 1 can quickly and easily evert a portion of a tissue structure by advancement of the bushing 4.

The radius of curvature of the everted portion can be greater than a value that would damage the tissue structure. The radius of curvature can be greater than 0.2 mm.

In an alternative example, the TTT 1 may include another mechanism to expand the leaves 2 and evert the portion 18. For example, the TTT 1 could include an outer ring that is connected to the leaves and slides backwards to pull the leaves outwards and then is fixed in place. In another alternative example, the TIT 1 may only retain the tissue structure and a separate device may be used to evert it. In a still further example, the widening process of FIGS. 17 and 18 below may be relied on to also evert the tissue structure.

In FIGS. 17 and 18, tool for widening a portion of a tubular tissue structure is shown. This may be useful when the tissue structure has a smaller diameter than that of the tissue structure it is to be coupled to. As shown in FIG. 17, the portion 18 of the tissue structure 16 is retained about an opening 17 of a TTT 1. Initially, the portion has a first diameter. A widening tool 40 is used to widen the opening 17, which also widens the retained portion 18 of the tissue structure 16 to have a second diameter. In the example of FIGS. 17 and 18, a tapered portion 41 of widening tool 40 is inserted into the opening 17 in the direction shown by arrows 43. The tool 40 may be gripped by an operator at the grip 42.

Insertion of the tapered portion 41 drives a plastically deformable portion of the bushing 4 radially outwards as indicated by the arrows 44 of FIG. 18. This drives the leaves 2 of the TTT 1 outwards. The bushing 4 can then hold this shape against an inward force of the leaves and tissue structure to retain the tissue structure in its widened state. The tissue structure can be widened to be closer to the width of another tissue structure that it is to be attached to. This can assist in coupling the tissue structures together. When the tissue structures to be coupled are of different diameters, whichever has the smaller diameter may be widened. In the case that the tissue structures have similar diameters, there may be no need for the widening process.

Alternatively, a different widening tool with an expandable portion may be used to widen the opening. Instead of the tapered portion, the expandable portion may be inserted into the opening and expanded to widen the opening. In one example, the tool includes a fluid enclosed by a deformable surface that can expand when the fluid is compressed in another region of the tool. As noted above, the expansion of the opening may also be used to evert the portion of the tissue structure by flexing the leaves outwards, thereby turning the retained portion 18 of the tissue structure outwards.

In FIGS. 19 to 21, TTTs 1 and 1′ are engaged with coupling devices 21 and 22 and tissue structures 16 and 16′ are coupled together using the coupling devices to form an anastomosis.

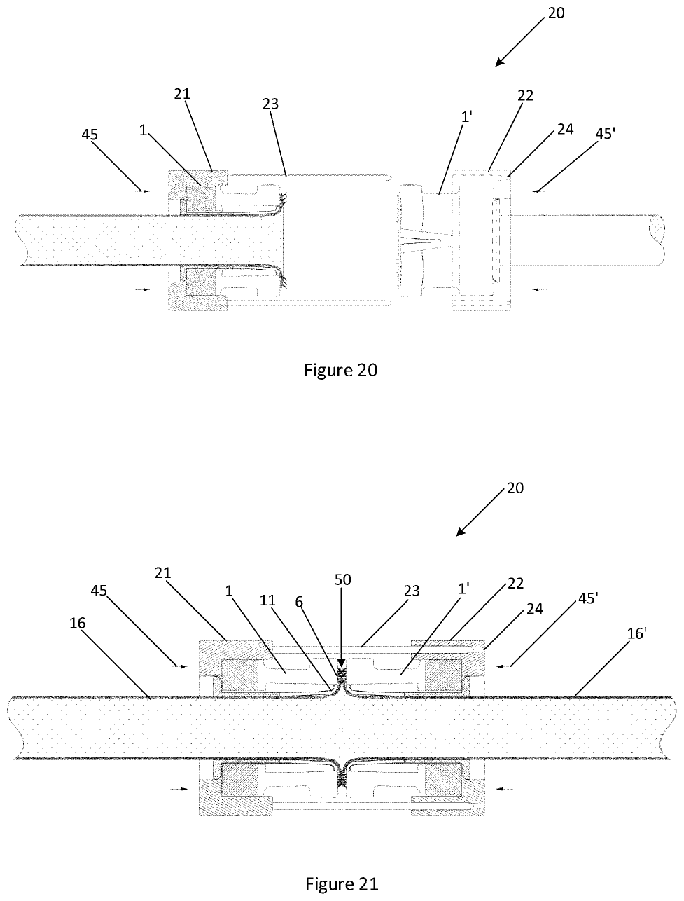

In FIG. 19, coupling devices 21 and 22 have been passed over the tissue structures 16, 16′ from the sides while everted portions of the tissue structures 16, 16′ are retained on the TTTs 1, 1′. The coupling devices 21, 22 are then moved towards their respective TTTs 1, 1′ in the directions indicated by the arrows 45 and 45′. This brings them into engagement with the TTTs 1, 1′ as shown in FIG. 20.

In FIG. 20, the TTTs 1, 1′ are engaged with the respective coupling devices 21, 22. In this example, the TTTs 1, 1′ are located in the coupling devices 21, 22 at predetermined relative angles about the longitudinal axis, as detailed with reference to FIG. 8. The coupling devices 21, 22, along with the TTTs 1, 1′, are then moved towards each other in the directions indicated by the arrows 45 and 45′ to bring them into engagement with each other and to bring the everted portions of the tissue structures 16, 16′ into contact with each other.

In FIG. 21, the coupling devices 21, 22 have been coupled to each other by way of pins 23 and holes 24. The everted portions of the tissue structures 16, 16′ have also been coupled to each other at interface 50 by being held against each other by the coupling devices 21, 22 without the need for sutures or staples.

As detailed with reference to FIG. 8, the TTTs 21, 22 are brought together with predetermined rotational offsets between the retainers of the TTTs. As detailed with reference to FIG. 3, the everted portions which are brought together are supported about substantially their whole perimeter. These features may ensure a good seal around the interface 50 and minimal leakage of fluid.

After coupling the devices 21, 22 and tissue structures 16, 16′ together, the operator may monitor the newly-formed anastomosis for leaks or other signs of poor coupling. If these are noticed, the operator may uncouple the devices 21, 22 by pulling them apart without the need to remove sutures or staples. This may be a non-destructive process so that the devices 21, 22 can be brought together again after being decoupled (and any other corrective action, such as reattachment, being taken) without the need to remove the TTTs 1, 1′ from the tissue structures 16, 16′ or cut off the retained portions of the tissue structures 16, 16′.

The described devices, systems and methods may allow quick, safe and easy attachment of tissue structures to tubular tissue transformers, eversion of tissue structures, widening of tissue structures and coupling of tissue structures with reduced risk of damage to the tissue structures, and which may be particularly suitable for coupling of arteries.

While the present invention has been illustrated by the description of the embodiments thereof, and while the embodiments have been described in detail, it is not the intention of the Applicant to restrict or in any way limit the scope of the appended claims to such detail. Additional advantages and modifications will readily appear to those skilled in the art. Therefore, the invention in its broader aspects is not limited to the specific details, representative apparatus and method, and illustrative examples shown and described. Accordingly, departures may be made from such details without departure from the spirit or scope of the Applicant's general inventive concept.

1. A tubular tissue transformer (TTT) for a tubular tissue structure, the tubular tissue transformer comprising: a plurality of leaves; and a plurality of retainers on or adjacent each of the plurality of leaves, each retainer configured to substantially retain the tubular tissue structure on the respective leaf.

2. The TTT of claim 1, wherein the plurality of retainers are pins.

3. The TTT of claim 2, wherein one or more of the pins are curved away from a longitudinal axis of the TTT.

4. The TTT of claim 2, wherein one or more of the pins are straight.

5. The TTT of claim 4, wherein one or more of the pins extend at an angle away from a longitudinal axis of the TTT.

6. -9. (canceled)

10. The TTT of claim 2, wherein each leaf has a plurality of rows of pins.

11. The TTT of claim 1, wherein one or more of the plurality of retainers each comprise a suction port, wherein the suction port is in communication with a source of low pressure via a suction line passing through the respective pin.

12.-15. (canceled)

16. The TTT of claim 1, wherein the TTT has a passage defined therethrough and wherein the plurality of leaves are located about an opening of the passage.

17. The TTT of claim 16, further comprising a bushing configured to be movable along the passage, wherein the bushing is configured to be movable along the passage from a first position to a second position to cause outward movement of a portion of each of the plurality of leaves.

18. (canceled)

19. The TTT of claim 17, wherein outward movement of the portion of the plurality of leaves is provided to evert the tubular tissue structure when retained by the retainers.

20. The TTT of claim 17, wherein the bushing has one or more support faces configured to support the outer surface of the tubular tissue structure in an everted state.

21. The TTT of claim 17, wherein the plurality of retainers comprises a plurality of leaf inserts, each leaf insert including one or more hooks to retain the tubular tissue structure on the leaf insert.

22. The TTT of claim 21, wherein the bushing is configured to interface with a base of each of the plurality of leaf inserts, whereby pushing the bushing longitudinally pushes the leaf inserts, which in turn cause outward movement of a portion of each of the plurality of leaves.

23.-29. (canceled)

30. A tubular tissue transformer (TTT) for a tubular tissue structure, the tubular tissue transformer comprising: a plurality of leaves configured to retain an everted portion of the tubular tissue structure; and a bushing configured to be movable between a first position and a second position and configured to support an outer surface of the everted portion of the tubular tissue structure in the second position.

31. (canceled)

32. The TTT of claim 30, wherein the bushing has one or more support faces configured to be located adjacent to the outer surface of the everted tubular tissue structure when the bushing is in the second position, wherein each of the one or more support faces curves outwardly by between 30° and 90°.

33.-35. (canceled)

36. The TTT of claim 32, wherein each of the plurality of leaves has a support face configured to be located adjacent to the outer surface of the everted portion of the tubular tissue structure.

37. The TTT of claim 36, wherein the support face of each of the plurality of leaves is configured to be located adjacent to, and at an angle of less than 45° to, a support face of the bushing when the bushing is in the second position.

38. (canceled)

39. The TTT of claim 30, wherein the bushing is configured to engage with the leaves to resist retraction away from the second position towards the first position.

40. (canceled)

41. The TTT of claim 30, configured such that the one or more leaves have a radially contracted configuration when the bushing is in the first position and a radially expanded configuration when the bushing is in the second position, wherein in the radially contracted configuration the leaves are configured to retain the tubular tissue structure in a less everted state than in the radially expanded configuration.

42. The TTT of claim 41, wherein the bushing has an outer surface arranged to contact an inner surface of the leaves in the second position to retain them in the expanded configuration.

43.-65. (canceled)
